(19) United States
(12) Patent Application Publication
(10) Pub. No.: US 2024/0091764 A1
(43) Pub. Date: Mar. 21, 2024

(54) COMBINABLE NUCLEIC ACID PRE-PROCESSING APPARATUS

(71) Applicant: Delta Electronics, Inc., Taoyuan City (TW)

(72) Inventors: Chien-Ting Liu, Taoyuan City (TW); Shih-Fang Peng, Taoyuan City (TW); Song-Bin Huang, Taoyuan City (TW); Guo-Wei Huang, Taoyuan City (TW); Jen-Chih Tsai, Taoyuan City (TW)

(21) Appl. No.: 18/370,181

(22) Filed: Sep. 19, 2023

Related U.S. Application Data
(60) Provisional application No. 63/408,394, filed on Sep. 20, 2022.

(51) Int. Cl. B01L 3/00

(52) U.S. Cl. CPC B01L 3/5027; B01L 2200/0621; B01L 2200/10; B01L 2300/0627; B01L 2300/0681; B01L 2300/0816; B01L 2300/0861; B01L 2300/1822; B01L 2400/0644

(57) ABSTRACT

A combinable nucleic acid pre-processing apparatus includes a sample transfer chamber transferring a sample from a sampling tube to a nucleic acid extraction kit, a nucleic acid extraction chamber performing a nucleic acid extraction of the sample in the nucleic acid extraction kit for obtaining a nucleic acid extract, an assay setup chamber preparing reagents and transferring reagents and the nucleic acid extract to an assay plate, and at least two bridging modules respectively disposed between the sample transfer chamber and the nucleic acid extraction chamber and between the nucleic acid extraction chamber and the assay setup chamber. The sample transfer chamber, the nucleic acid extraction chamber and the assay setup chamber are separated and operated independently. Three chambers are connected through the bridging modules, so the nucleic acid extraction kit can be sequentially moved in the sample transfer chamber, the nucleic acid extraction chamber and the assay setup chamber.

### CROSS-REFERENCE TO RELATED APPLICATION

This application claims the benefit of U.S. Provisional Application No. 63/408,394 filed on Sep. 20, 2022 and entitled “COMBINABLE NUCLEIC ACID DETECTION SYSTEM”. The entireties of the above-mentioned patent application are incorporated herein by reference for all purposes.

### FIELD OF THE INVENTION

The present disclosure relates to a combinable nucleic acid pre-processing apparatus, and more particularly to a combinable nucleic acid pre-processing apparatus including multiple combinable independent chambers.

### BACKGROUND OF THE INVENTION

The growth of global population and the development of modern transportation have not only greatly improved international trade and economic development, but also intensified the spread of infectious diseases. Especially during the global pandemic of coronavirus disease 2019 (COVID-19), the widely spread virus not only affected the global economy, but also caused a lot of inconvenience to human life. The polymerase chain reaction (PCR) detection has been widely used with the COVID-19 pandemic. However, the conventional PCR detection requires time and labor in sample processing, nucleic acid extraction, and subsequent amplification and detection processes.

For example, before detecting nucleic acids in biological samples, multiple processing procedures, e.g., reading barcodes on the sampling tubes, opening and closing covers of the sampling tubes, transferring samples, extracting nucleic acids, and preparing reagents, are required and involve many operation steps, most of which still have to be done manually. Therefore, the detection efficiency and the detection accuracy are influenced, and the possibility of cross contamination between samples and the risk of operator infection are also increased.

Currently, automated apparatuses for collectively performing the pre-processing procedures of nucleic acids are developed. However, in the developed automated apparatus, the processing of samples is by batch, namely, the next batch of samples is processed only after the processing of the current batch of samples is completed. Thus, the throughput per unit time is limited.

Therefore, there is a need of providing a combinable nucleic acid pre-processing apparatus for improving the throughput per unit time.

### SUMMARY OF THE INVENTION

An object of the present disclosure is to provide a combinable nucleic acid pre-processing apparatus which is separated into multiple chambers through utilizing bridging modules. Each chamber can perform partial procedures of the nucleic acid pre-processing process independently, and the chambers also can be connected to cooperatively complete the entire nucleic acid pre-processing process.

Another object of the present disclosure is to provide a combinable nucleic acid pre-processing apparatus, in which the nucleic acid pre-processing process is separated for being performed in different chambers, so that each chamber can perform partial pre-processing procedures for different batches of samples, thereby significantly improving the throughput per unit time.

A further object of the present disclosure is to provide a combinable nucleic acid pre-processing apparatus which can automatically perform all procedures after samples are obtained and before PCR detection is performed, which effectively reduces the labor requirement.

In accordance with an aspect of the present disclosure, a combinable nucleic acid pre-processing apparatus is provided. The combinable nucleic acid pre-processing apparatus includes a sample transfer chamber, a nucleic acid extraction chamber, an assay setup chamber and at least two bridging modules. The sample transfer chamber includes a cover opening and closing module and a first pipetting module for transferring a sample from a sampling tube to a nucleic acid extraction kit. The nucleic acid extraction chamber includes a nucleic acid extraction module for performing a nucleic acid extraction operation on the sample in the nucleic acid extraction kit so as to obtain a nucleic acid extract. The assay setup chamber includes a second pipetting module for deploying a reagent and adding the deployed reagent and the nucleic acid extract into an assay plate. The at least two bridging modules are respectively disposed between the sample transfer chamber and the nucleic acid extraction chamber and between the nucleic acid extraction chamber and the assay setup chamber. The sample transfer chamber, the nucleic acid extraction chamber and the assay setup chamber are separated from each other and operated independently, and the sample transfer chamber, the nucleic acid extraction chamber and the assay setup chamber are further connected through the at least two bridging modules for achieving a sequential movement of the nucleic acid extraction kit in the sample transfer chamber, the nucleic acid extraction chamber and the assay setup chamber in a duration of nucleic acid pre-processing.

In an embodiment, the sample transfer chamber, the nucleic acid extraction chamber and the assay setup chamber individually perform nucleic acid pre-processing procedures of different batches of samples simultaneously.

In an embodiment, each bridging module includes a first bridging structure and a second bridging structure respectively disposed on adjacent sides of adjacent two chambers, and by combining the first bridging structure and the second bridging structure, the adjacent two chambers are connected to each other.

In an embodiment, the first bridging structure includes a first engaging member, a first opening, a first partition board and a first sliding rail, and the first partition board moves along the first sliding rail to open or close the first opening. The second bridging structure includes a second engaging member, a second opening, a second partition board and a second sliding rail, and the second partition board moves along the second sliding rail to open or close the second opening.

In an embodiment, the first engaging member and the second engaging member are engaged to form a connection channel between the adjacent two chambers for the nucleic acid extraction kit to pass therethrough, the first partition board and the second partition board are located at two opposite ends of the connection channel, and through closing and opening the first opening and the second opening, the adjacent two chambers are mutually separated or connected.

In an embodiment, the first bridging structure includes a first protrusion board for forming an inverted trapezoidal shape space around the first opening, the first partition board includes a second protrusion board in an inverted trapezoidal shape, airtight elements are disposed at a periphery of the second protrusion board, and when the first partition board is moved downwardly, the second protrusion board enters the inverted trapezoidal shape space, and wherein the second bridging structure includes a third protrusion board for forming an inverted trapezoidal shape space around the second opening, the second partition board includes a fourth protrusion board in an inverted trapezoidal shape, airtight elements are disposed at a periphery of the fourth protrusion board, and when the second partition board is moved downwardly, the fourth protrusion board enters the inverted trapezoidal shape space.

In an embodiment, the sample transfer chamber includes a first nucleic acid extraction kit conveying module for conveying the nucleic acid extraction kit to the nucleic acid extraction chamber.

In an embodiment, the sample transfer chamber includes a first air inlet, a first air outlet and a fan filter unit disposed at the first air outlet for maintaining a first airflow entering the sample transfer chamber in a single direction, and the first airflow does not pass through an area where the nucleic acid extraction kit is placed after passing through an uncovered sampling tube, and wherein the first air inlet and the first air outlet respectively have high efficiency particulate air filters disposed thereon.

In an embodiment, the nucleic acid extraction module includes a magnetic rod holder, a plurality of magnetic rods mounted on the magnetic rod holder, a magnetic rod sleeve connector holder, and a plurality of magnetic rod sleeve connectors mounted on the magnetic rod sleeve connector holder for performing a magnetic beads-based nucleic acid extraction operation.

In an embodiment, the nucleic acid extraction chamber includes a second nucleic acid extraction kit conveying module for receiving the nucleic acid extraction kit from the sample transfer chamber and for conveying the nucleic acid extraction kit to the assay setup chamber.

In an embodiment, the nucleic acid extraction chamber includes a temperature controlling module for matching with each well of the nucleic acid extraction kit so as to provide a temperature variation required during the nucleic acid extraction operation.

In an embodiment, the nucleic acid extraction chamber includes a second air inlet, a second air outlet, a first deflector, a second deflector and a fan filter unit disposed at the second air outlet for maintaining a second airflow entering the nucleic acid extraction chamber in a single direction, and the second airflow downwardly passes through an area for performing the nucleic acid extraction operation, and wherein the second air inlet and the second air outlet respectively have high efficiency particulate air filters disposed thereon.

In an embodiment, the assay setup chamber includes a third nucleic acid extraction kit conveying module for receiving the nucleic acid extraction kit from the nucleic acid extraction chamber.

In an embodiment, the assay setup chamber includes an assay plate conveying module and an assay plate sealing module, the assay plate conveying module conveys the assay plate with the added reagent and the added nucleic acid extract to the assay plate sealing module, and the assay plate sealing module performs a film sealing procedure to the assay plate.

In an embodiment, the assay setup chamber includes a third air inlet, a third air outlet, a third deflector and a fan filter unit disposed at the third air outlet for maintaining a third airflow entering the assay setup chamber in a single direction, and the third airflow flows toward an area where the nucleic acid extraction kit is placed. The third air inlet and the third air outlet respectively have high efficiency particulate air filters disposed thereon.

The above contents of the present disclosure will become more readily apparent to those ordinarily skilled in the art after reviewing the following detailed description and accompanying drawings, in which:

### BRIEF DESCRIPTION OF THE DRAWINGS

FIG. 1 is a schematic view showing a combinable nucleic acid pre-processing apparatus of the present disclosure;

FIG. 2 a perspective top view showing the combinable nucleic acid pre-processing apparatus of the present disclosure;

FIG. 3A is a schematic view showing a sample transfer chamber of the present disclosure;

FIG. 3B is a schematic view showing the sample transfer chamber of the present disclosure from another view angle;

FIG. 4A is a schematic view showing a nucleic acid extraction chamber of the present disclosure;

FIG. 4B is a schematic view showing the nucleic acid extraction chamber of the present disclosure from another view angle;

FIG. 5A is a schematic view showing an assay setup chamber of the present disclosure;

FIG. 5B is a schematic view showing the assay setup chamber of the present disclosure from another view angle;

FIG. 6A is a schematic view showing a first bridging structure of the present disclosure;

FIG. 6B is a schematic view showing the first bridging structure of the present disclosure from another view angle;

FIG. 7A is a schematic view showing a second bridging structure of the present disclosure;

FIG. 7B is a schematic view showing the second bridging structure of the present disclosure from another view angle;

FIG. 8A is a front view showing the first bridging structure when a first partition board is at an opening position of the present disclosure;

FIG. 8B is a front view showing a side of the first partition board facing a first opening of the first bridging structure of the present disclosure;

FIG. 8C is a side view of the first partition board of the present disclosure;

FIG. 9 is a schematic view showing portions of structures of the sample transfer chamber of the present disclosure;

FIG. 10 is a schematic view showing a cover opening and closing module and a sampling tube visual identification module of the present disclosure;

FIG. 11 is a schematic view showing a first pipetting module and a first pipetting visual identification module of the present disclosure;

FIG. 12A is a schematic view showing a first nucleic acid extraction kit conveying module of the present disclosure;

FIG. 12B is schematic view showing the first nucleic acid extraction kit conveying module having nucleic acid extraction kits disposed thereon of the present disclosure;

FIG. 13 is a perspective side view of the sample transfer chamber of the present disclosure;

FIG. 14 is a schematic view showing portions of structures of the nucleic acid extraction chamber of the present disclosure;

FIG. 15 is a schematic view showing a nucleic acid extraction module of the present disclosure;

FIG. 16A is a schematic view showing a second nucleic acid extraction kit conveying module and a temperature controlling module of the present disclosure;

FIG. 16B is schematic view showing the second nucleic acid extraction kit conveying module having nucleic acid extraction kits disposed thereon of the present disclosure;

FIG. 17 is a perspective side view of the nucleic acid extraction chamber of the present disclosure;

FIG. 18A is a schematic view showing portions of structures of the assay setup chamber of the present disclosure;

FIG. 18B is a schematic view showing portions of structures of the assay setup chamber of the present disclosure from another view angle;

FIG. 19 is a schematic view showing a second pipetting module and a second pipetting visual identification module of the present disclosure;

FIG. 20 is a schematic view showing an assay plate conveying module of the present disclosure;

FIG. 21A is a schematic view showing a third nucleic acid extraction kit conveying module of the present disclosure;

FIG. 21B is a schematic view showing the third nucleic acid extraction kit conveying module having nucleic acid extraction kits disposed thereon of the present disclosure; and

FIG. 22 is a perspective side view of the assay setup chamber of the present disclosure.

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for purpose of illustration and description only. It is not intended to be exhaustive or to be limited to the precise form disclosed. The drawings of all the embodiments of the present disclosure are merely schematic and do not represent true dimensions and proportions.

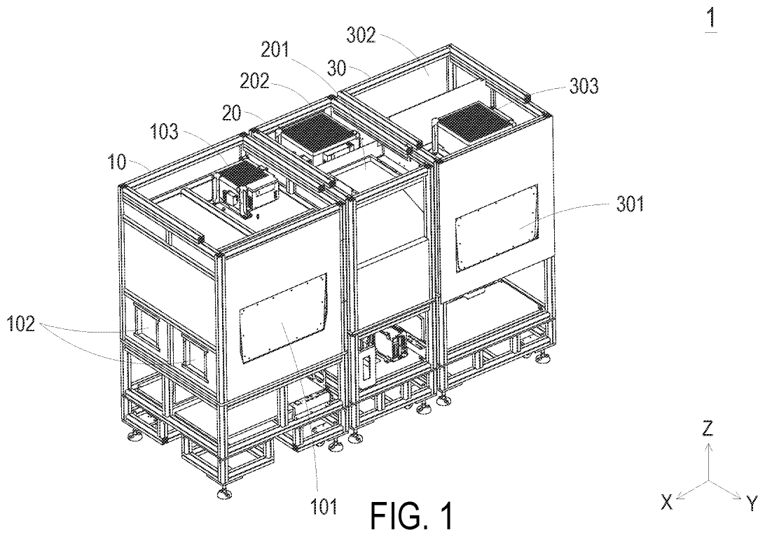

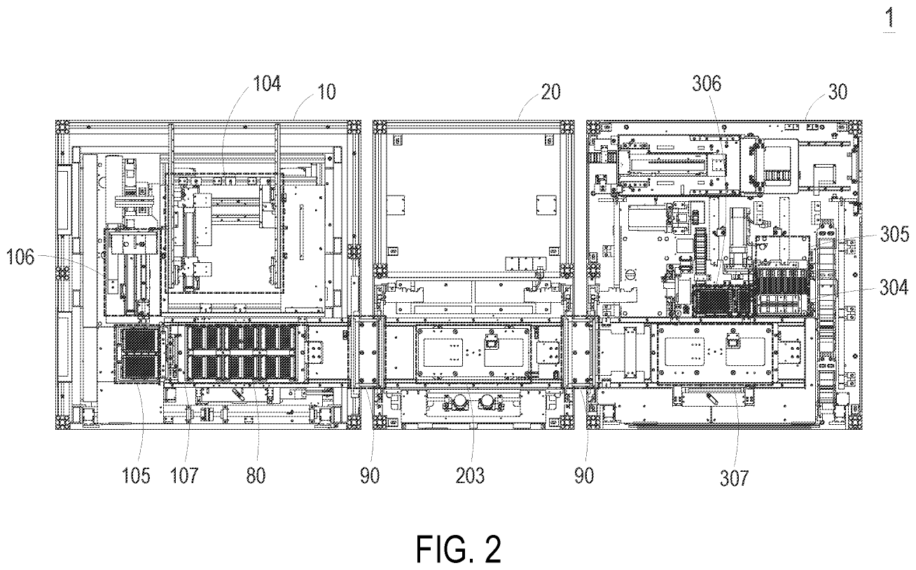

Please refer to FIG. 1 and FIG. 2. FIG. 1 is a schematic view showing a combinable nucleic acid pre-processing apparatus of the present disclosure, and FIG. 2 a perspective top view showing the combinable nucleic acid pre-processing apparatus of the present disclosure. A combinable nucleic acid pre-processing apparatus 1 includes three chambers which respectively are a sample transfer chamber 10, a nucleic acid extraction chamber 20 and an assay setup chamber 30. The three chambers 10, 20, 30 are capable of being separated from each other and also capable of being mechanically combined together from left to right as shown in FIG. 2. The three chambers 10, 20, 30 respectively are used to perform partial procedures in a nucleic acid pre-processing process independently, and when mechanically combined together, the three chambers 10, 20, 30 can cooperatively complete the entire nucleic acid pre-processing process.

In the combinable nucleic acid pre-processing apparatus 1, a plurality of pre-processing procedures of the nucleic acid pre-processing process are separated into three groups in accordance with properties thereof and respectively performed in the three chambers 10, 20, 30. The nucleic acid extraction chamber 20 performs the nucleic acid extraction, such as the magnetic beads-based nucleic acid extraction, for obtaining a nucleic acid extract from a sample. The sample transfer chamber 10 performs procedures before the nucleic acid extraction, including but not limited to opening and closing covers of sampling tubes, identifying barcodes on sampling tubes, and transferring samples from sampling tubes. The assay setup chamber 30 performs procedures after the nucleic acid extraction and before the nucleic acid detection, including but not limited to preparing reagents and transferring nucleic acid extracts. Through this scheme, the operation of nucleic acid extraction which might easily cause cross-contamination is isolated in an independent space, and by cooperating with a nucleic acid extraction kit which has magnetic rod sleeves pre-set therein and reagents pre-loaded therein, the nucleic acid extraction can be operated automatically in the chamber without manually loading and unloading consumables, which not only significantly reduces the labor and time of manual operation, but also avoids the cross-contamination caused by consumables with liquids attached thereon.

Based on the design that each chamber operates independently, three chambers can separately and independently perform the procedures thereof at the same time, and after completing the procedures and forwarding the current batch of samples to the next chamber for subsequent procedures, the chamber can immediately perform the procedures for the next batch of samples without waiting the subsequent procedures of the current batch of samples to be completed. Therefore, all chambers can individually and simultaneously perform procedures in the nucleic acid pre-processing process for different batches of samples, so the throughput per unit time can be significantly improved. For example, after the sample transfer chamber 10 receives a first batch of samples and completes the sample transfer procedures thereof, the first batch of samples is moved to the nucleic acid extraction chamber 20 for performing the nucleic acid extraction operation, and at the same time, the sample transfer chamber 10 receives a second batch of samples and performs the sample transfer procedures thereof after being disinfected. Thus, the sample transfer chamber 10 and the nucleic acid extraction chamber 20 are operated individually and simultaneously. Then, after the nucleic acid extraction operation of the first batch of samples in the nucleic acid extraction chamber 20 is completed, the first batch of samples is moved to the assay setup chamber 30 to perform the subsequent assay setup procedures, and at the same time, the sample transfer procedures of the second batch of samples in the sample transfer chamber 10 are also completed, so the nucleic acid extraction chamber 20 receives the second batch of samples to perform the nucleic acid extraction operation after being disinfected, and the sample transfer chamber 10 receives a third batch of samples to perform the sample transfer procedures after the second batch of samples is moved out and the disinfection is also done. Thus, the sample transfer chamber 10, the nucleic acid extraction chamber 20 and the assay setup chamber 30 are operated at the same time. In other words, other than the first and the second batches of samples, starting from the third batch of samples, the three chambers can operate individually and simultaneously to perform the partial pre-processing procedures of different batches of samples continuously without idling to wait for all pre-processing procedures of the single batch of samples to be completed, which effectively improves the throughput per unit time.

Moreover, because the pre-processing procedures are separated into three chambers, if the operation of individual chamber is abnormal, it only needs to troubleshoot the abnormality in the abnormal chamber without influencing other chambers. Furthermore, since the entire apparatus is divided into three independent chambers, the volume of each chamber becomes smaller, and the handling, transportation, and installation thereof also become easier.

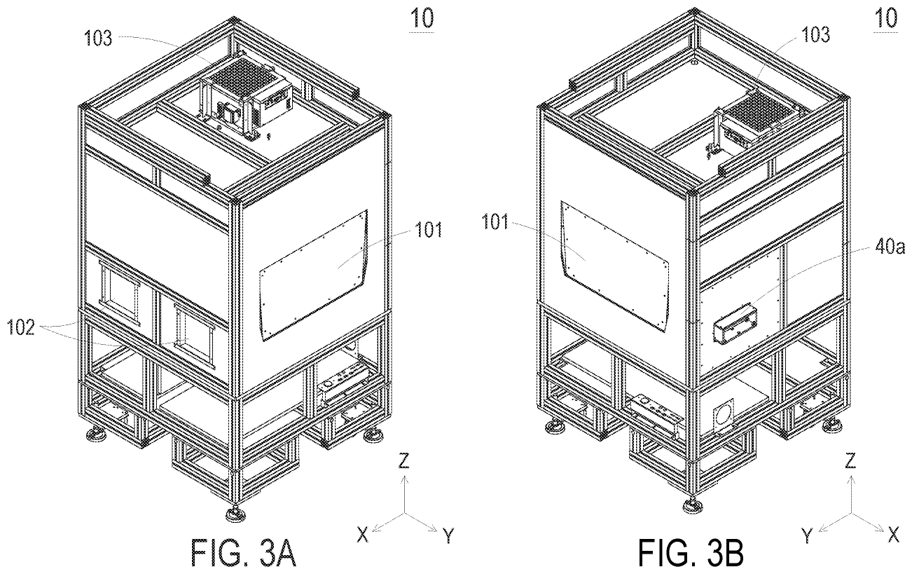

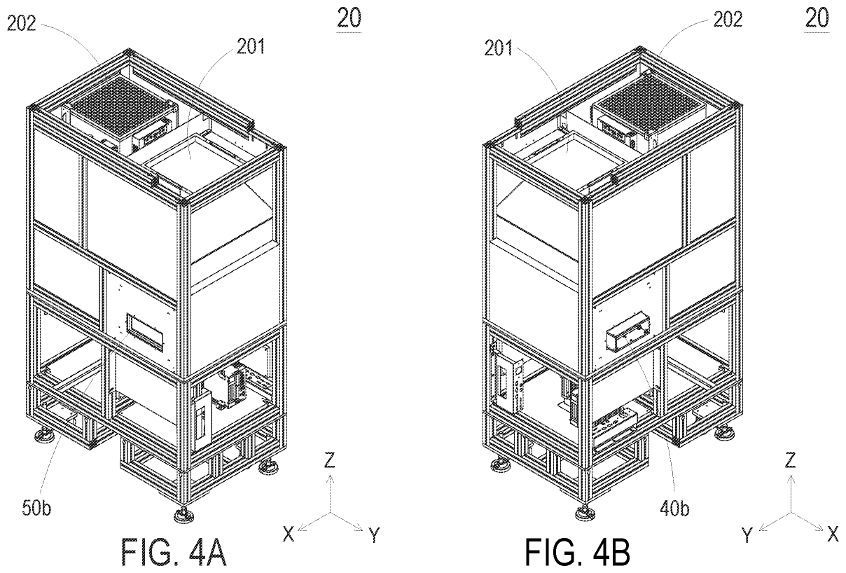

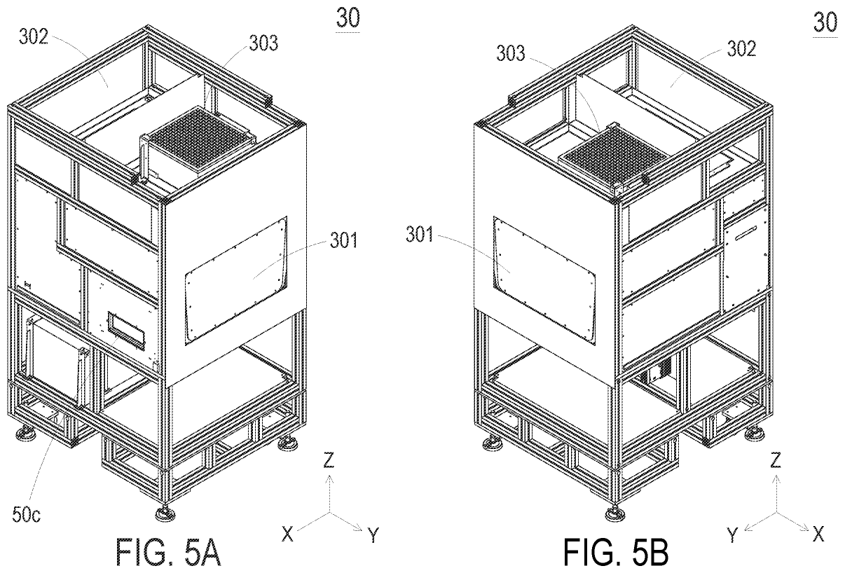

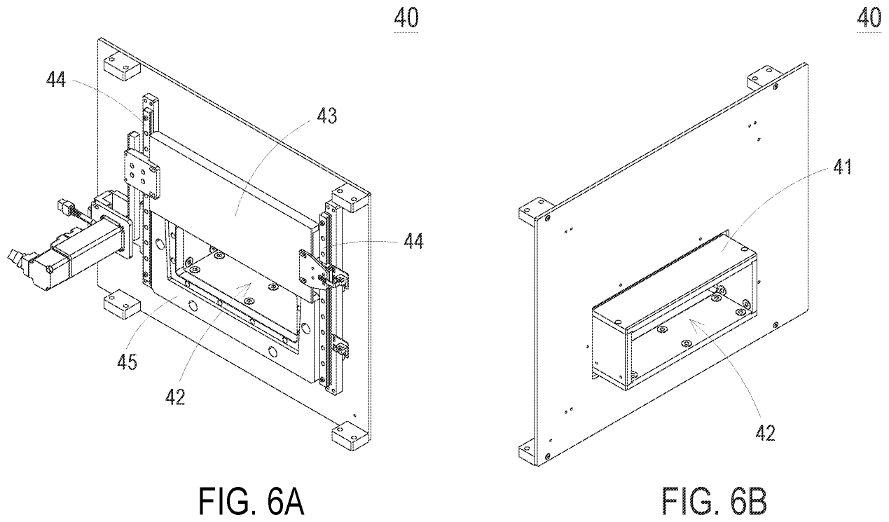

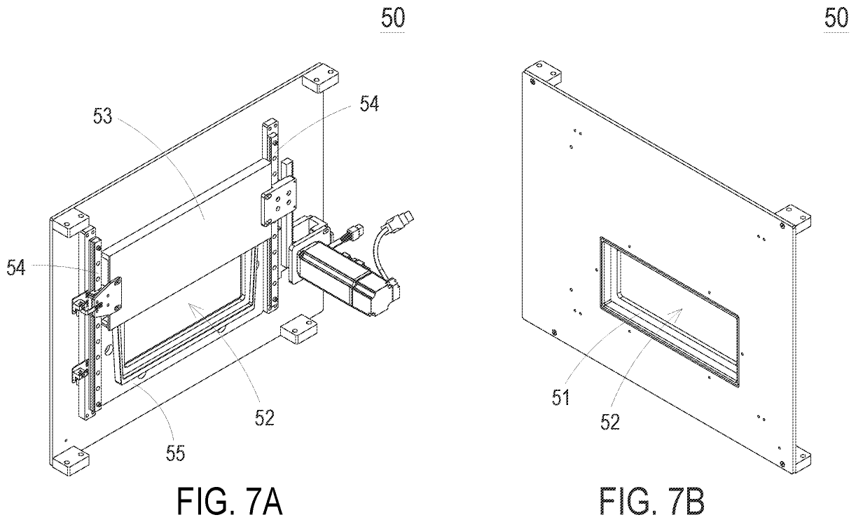

The key for achieving that three chambers are capable of operating individually and cooperatively is bridging modules disposed among the chambers. Please refer to FIG. 2, FIGS. 3A-3B, FIGS. 4A-4B, FIGS. 5A-5B, FIGS. 6A-6B and FIGS. 7A-7B. FIG. 3A is a schematic view showing a sample transfer chamber of the present disclosure, FIG. 3B is a schematic view showing the sample transfer chamber of the present disclosure from another view angle, FIG. 4A is a schematic view showing a nucleic acid extraction chamber of the present disclosure, FIG. 4B is a schematic view showing the nucleic acid extraction chamber of the present disclosure from another view angle, FIG. 5A is a schematic view showing an assay setup chamber of the present disclosure, FIG. 5B is a schematic view showing the assay setup chamber of the present disclosure from another view angle, FIG. 6A is a schematic view showing a first bridging structure of the present disclosure, FIG. 6B is a schematic view showing the first bridging structure of the present disclosure from another view angle, FIG. 7A is a schematic view showing a second bridging structure of the present disclosure, and FIG. 7B is a schematic view showing the second bridging structure of the present disclosure from another view angle. The combinable nucleic acid pre-processing apparatus 1 includes at least two bridging modules respectively disposed between the sample transfer chamber 10 and the nucleic acid extraction chamber 20, and between the nucleic acid extraction chamber 20 and the assay setup chamber 30, namely, adjacent two chambers has at least one bridging module disposed therebetween. Each bridging module includes a first bridging structure 40 and a second bridging structure 50. The first bridging structure 40 includes a first engaging member 41, a first opening 42, a first partition board 43 and a first sliding rail 44, and the second bridging structure 50 includes a second engaging member 51, a second opening 52, a second partition board 53 and a second sliding rail 54. The first engaging member 41 and the second engaging member 51 are mutually engaged for achieving the connection and fixation between adjacent two chambers. In an embodiment, the first engaging member 41 and the second engaging member 51 are respectively a protrusion member and a recess member which are correspondingly disposed and capable of engaging with each other, and through the protrusion member extends into the recess member in a manner that the outer periphery of the protrusion member closely fit to the inner periphery of the recess member, a combination therebetween can be achieved. The first engaging member 41 and the second engaging member 51 can be implemented as any kind of structures which are capable of engaging mutually, and are not limited thereto. The first partition board 43 and the second partition board 53 provide functions of airtightness and separation, wherein the first partition board 43 and the second partition board 53 can be slid respectively along the first sliding rail 44 and the second sliding rail 54 to move up to expose the first opening 42 and the second opening 52 and move down to close the first opening 42 and the second opening 52. Therefore, through disposing the first bridging structure 40 and the second bridging structure 50, the connection between adjacent two chambers can be achieved conveniently without using screws, and at the same time, the airtightness is also achieved.

In an embodiment, the sample transfer chamber 10 has a first bridging structure 40a disposed at the side facing the nucleic acid extraction chamber 20 and the nucleic acid extraction chamber 20 correspondingly has a second bridging structure 50b disposed at the side facing the sample transfer chamber 10, and the nucleic acid extraction chamber 20 has a first bridging structure 40b disposed at the side facing the assay setup chamber 30 and the assay setup chamber 30 correspondingly has a second bridging structure 50c disposed at the side facing the nucleic acid extraction chamber 20, so three chambers can be combined together. Of course, in accordance with different demands, there also can have a plurality of bridging modules disposed between adjacent two chambers without being limited thereto. For example, a plurality of bridging modules can be disposed between the sample transfer chamber 10 and the nucleic acid extraction chamber 20, namely, a plurality of first bridging structures and a plurality of corresponding second bridging structures are respectively disposed on the sample transfer chamber 10 and the nucleic acid extraction chamber 20; and/or a plurality of bridging modules can be disposed between the nucleic acid extraction chamber 20 and the assay setup chamber 30, namely, a plurality of first bridging structures and a plurality of corresponding second bridging structures are respectively disposed on the nucleic acid extraction chamber 20 and the assay setup chamber 30.

After the first bridging structure 40 and the second bridging structure 50 are connected, a connection channel 90 is formed between adjacent two chambers, and the first partition board 43 and the second partition board 53 are disposed at two opposite ends of the connection channel 90. When the partition boards at two ends are opened, the adjacent chambers can be communicated through the connection channel 90, and when the partition boards at two ends are closed, the adjacent chambers are separated as two independent spaces.

As described above, the design of three chambers divides the nucleic acid pre-processing process into three portions, including procedures of transferring samples to the nucleic acid extraction kit, procedures of performing the nucleic acid extraction operation in the nucleic acid extraction kit, and procedures of transferring nucleic acid extracts from the nucleic acid extraction kit to the assay plate for detection. Since the procedures performed in each chamber still have continuity and the samples have to move sequentially in the sample transfer chamber 10, the nucleic acid extraction chamber 20 and the assay setup chamber 30 to complete the entire nucleic acid pre-processing process, under this premise, the present disclosure utilizes the nucleic acid extraction kit 80 as the transportation medium for the same batch of samples to move sequentially in three chambers. Therefore, the connection channel 90 formed by the bridging module is meant to let the nucleic acid extraction kit 80 pass therethrough, and accordingly, the entire nucleic acid pre-processing process can be performed in the combinable nucleic acid pre-processing apparatus 1, which is in a combined state.

When the samples in the sampling tubes are transferred to the nucleic acid extraction kit 80 in the sample transfer chamber 10, the first partition board 43 of the first bridging structure 40a of the sample transfer chamber 10 is opened and the second partition board 53 of the second bridging structure 50b of the nucleic acid extraction chamber 20 is opened, so the nucleic acid extraction kit 80 in the sample transfer chamber 10 can pass through the connection channel 90 formed by the first engaging member 41 and the second engaging member 51 and enter the nucleic acid extraction chamber 20. Then, the first partition board 43 and the second partition board 53 are closed, and the sample transfer chamber 10 and nucleic acid extraction chamber 20 are separated again as independent spaces. After the nucleic acid extraction operation in the nucleic acid extraction chamber 20 is completed, the first partition board 43 of the first bridging structure 40b at the opposite side of the nucleic acid extraction chamber 20 is opened and the second partition board 53 of the second bridging structure 50c of the assay setup chamber 30 is opened, so the nucleic acid extraction kit 80 in the nucleic acid extraction chamber 20 can pass through the connection channel 90 formed by the first engaging member 41 and the second engaging member 51 and enter the assay setup chamber 30. Then, the first partition board 43 and the second partition board 53 are closed, and the nucleic acid extraction chamber 20 and the assay setup chamber 30 are separated again as independent spaces. Finally, the assay setup chamber 30 is operated to transfer the nucleic acid extracts in the nucleic acid extraction kit 80 to the assay plate for further detection. In addition, the first bridging structure and/or the second bridging structure of each chamber are disposed at the same horizontal plane, so the nucleic acid extraction kit 80 can be moved in the same height in Z axis direction.

Consequently, in the combinable nucleic acid pre-processing apparatus 1 of the present disclosure, through the bridging modules, even though the procedures are separated and performed in different chambers, the entire process still can be completed automatically and sequentially in the chambers.

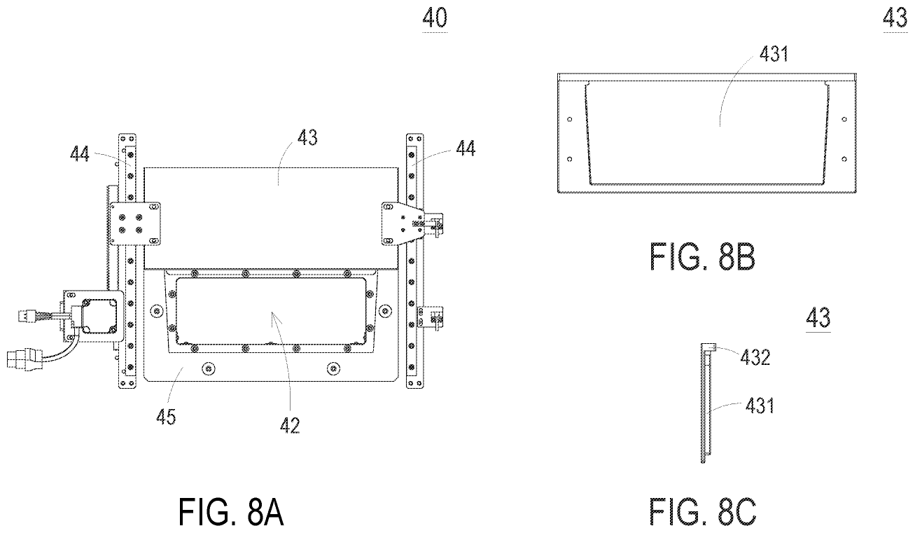

Please refer to FIGS. 6A-6B, FIGS. 7A-7B and FIGS. 8A-8B. FIG. 8A is a front view showing the first bridging structure when the first partition board is at an opening position of the present disclosure, FIG. 8B is a front view showing the side of the first partition board facing the first opening of the first bridging structure of the present disclosure, and FIG. 8C is a side view of the first partition board of the present disclosure. The first bridging structure 40 further includes a first protrusion board 45 disposed between the first opening 42 and the first partition board 43. The first protrusion board 45 is protruded and disposed at the periphery of the lower edge of the first opening 42 and two sides, which are connected to the lower edge, namely, the first protrusion board 45 surrounds three sides of the first opening 42. Moreover, the first partition board 43 has a second protrusion board 431 disposed at a surface facing the first opening 42 and corresponding to the first protrusion board 45.

For maintaining the airtightness of each chamber, the second protrusion board 431 is designed to have an inverted trapezoidal shape, namely, the lower edge and the upper edge are parallel to each other and the width of the upper edge is larger than that of the lower edge, and further, the first protrusion board 45 is designed to form an inverted trapezoidal shape space at three sides of the first opening 42 which has an identical shape to the second protrusion board 431 but is in a larger size. Therefore, the second protrusion board 431 can be accommodated in the first protrusion board 45. Moreover, the lower edge of the second protrusion board 431 and two opposite sides connected to the lower edge and the upper edge respectively have an airtight element (not shown) disposed thereon. Through adopting the inverted trapezoidal shape, when the first opening 42 is opened, the lower edge of the inverted trapezoidal shape of the second protrusion board 431 is approximately located at the upper edge of the inverted trapezoidal shape formed by the first protrusion board 45, so even though there are airtight elements disposed on two opposite sides thereof, the second protrusion board 431 still can slide along the first sliding rail 44 to move down and enter the inverted trapezoidal shape space formed by the first protrusion board 45. Then, since the width of the inverted trapezoidal shape space gradually decreases from the upper edge to the lower edge, the airtight elements are gradually compressed, the second protrusion board 431 is embedded in the first protrusion board 45, and the airtight elements are averagely embedded between the inner periphery of the first protrusion board 45 and the two opposite sides and the lower edge of the second protrusion board 431, thereby achieving the airtightness effect. Moreover, the first partition board 43 further includes a protrusion edge 432 disposed at the top of the second protrusion board 431, and the lower surface of the protrusion edge 432 also has an airtight element (not shown) disposed thereon, so when the first partition board 43 is moved down to close the first opening 42, the airtight element can be embedded between the protrusion edge 432 and the upper edge of the first opening 42 for further ensuring the airtightness effect.

In the second bridging structure 50, the manner to achieve the airtightness are the same with the first bridging structure 40, and the shape of the second partition board 53 is also identical to that of the first partition board 43. That is, the periphery of the second opening 52 has a third protrusion board 55 to form the inverted trapezoidal shape space, and the second partition board 53 has a fourth protrusion board (not shown) in the inverted trapezoidal shape, so when the second partition board 53 is moved down to close the second opening 52, the fourth board can enter the inverted trapezoidal shape space and airtight elements (not shown) disposed around the fourth protrusion board can achieve the airtightness effect. Other details are similar and omitted.

On the other hand, the configuration of each chamber is also designed to reduce the possibility of contamination. First, there are airtight elements and sealing boards disposed in each chamber, and a fan filter unit (FFU) is also disposed at an air outlet of each chamber, so as to maintain a slight negative pressure in each chamber. Accordingly, even though the airtightness of the chamber is not good enough, the risk of contamination caused by the air inside the chamber flowing out still can be suppressed. Moreover, high efficiency particulate air (HEPA) filters are disposed at air inlets and air outlets of each chamber for ensuring that the air flowed in and out of each chamber is filtered to be clean so as to reduce the risk of contamination. In an embodiment, it can be implemented to replace the air in the chamber more than once per minute. Furthermore, each chamber is equipped with UV irradiations (e.g., 239 nm UV) for disinfecting. For example, UV irradiations can be performed to clean the entire chamber when the chamber starts up, to clean the local area after the performed procedures end, and/or to re-clean the chamber when the chamber has been left standing for too long (such as more than 30 minutes). In addition, after the pre-processing procedures of each batch of samples have been completed, other than UV irradiations, the air in each chamber also will be replaced more than once for avoiding the cross-contamination between different batches.

For avoiding the cross-contamination, when the partition boards 43, 53 of the bridging modules between adjacent two chambers are opened, the principle is the air is guided to flow to the nucleic acid extraction chamber 20 which has a high contamination risk. For example, when the nucleic acid extraction kit 80 is moved from the sample transfer chamber 10 to the nucleic acid extraction chamber 20, the FFU of the sample transfer chamber 10 is turned off and the FFU of the nucleic acid extraction chamber 20 remains in operation, so as to ensure the air does not flow from the nucleic acid extraction chamber 20 with high contamination risk to the sample transfer chamber 10. When the nucleic acid extraction kit 80 is moved from the nucleic acid extraction chamber 20 to the assay setup chamber 30, the FFU of the nucleic acid extraction chamber 20 remains in operation and the FFU of the assay setup chamber 30 is turned off, so as to ensure the air does not flow from the nucleic acid extraction chamber 20 with high contamination risk to the assay setup chamber 30. Besides, the principle as planning the airflow in each chamber is the air first passes through an area with low contamination risk, such as, an area where the consumables or the reagents are placed, and then passes through an area with high contamination risk, thereby reducing the occurrence of cross contamination. Since each chamber is utilized to perform different pre-processing procedures, the positions of the air inlet and the air outlet of each chamber are correspondingly different, and there also has deflector(s) disposed inside the chamber for achieving the airflow planning Details are described with each chamber below.

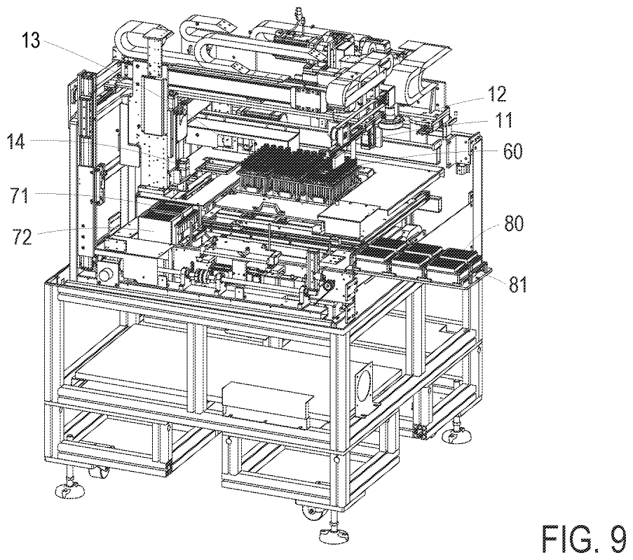

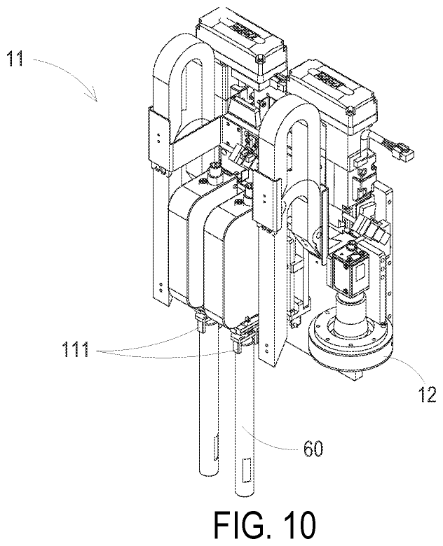

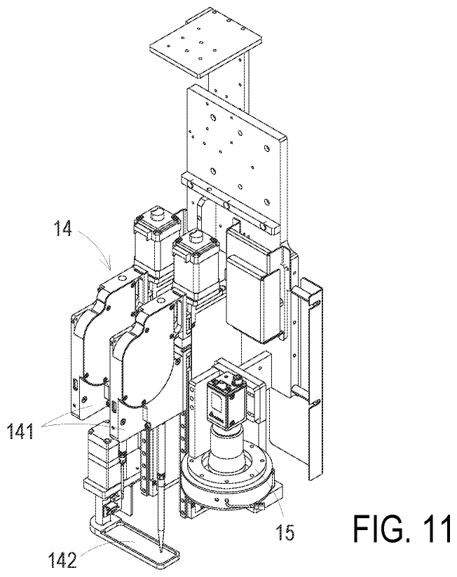

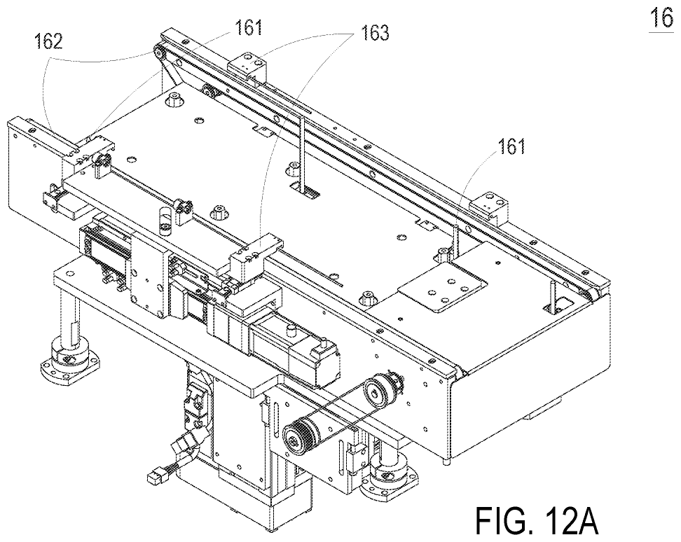

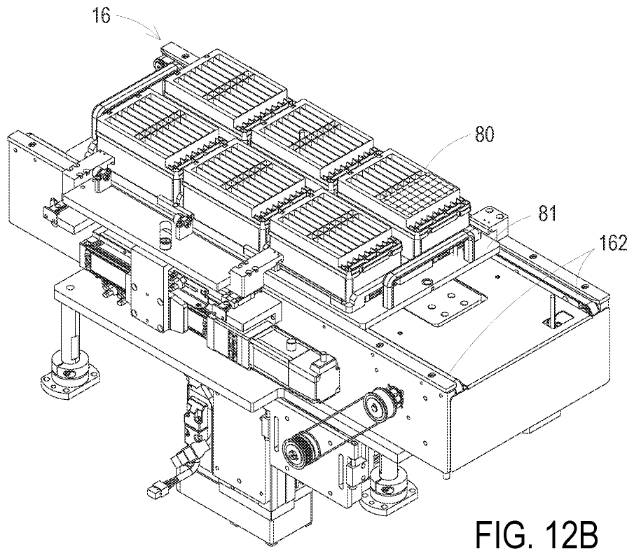

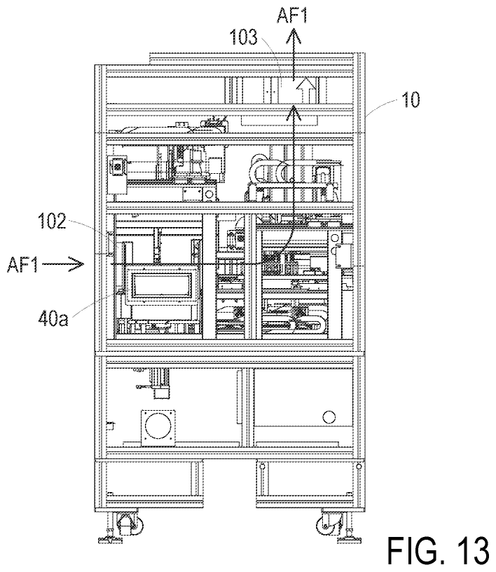

Please refer to FIG. 1, FIG. 2, FIGS. 3A-3B, FIGS. 9-11, FIGS. 12A-12B and FIG. 13. FIG. 9 is a schematic view showing portions of structures of the sample transfer chamber of the present disclosure, FIG. 10 is a schematic view showing a cover opening and closing module and a visual identification module of the present disclosure, FIG. 11 is a schematic view showing a first pipetting module and a first pipetting visual identification module of the present disclosure, FIG. 12A is a schematic view showing a first nucleic acid extraction kit conveying module of the present disclosure, FIG. 12B is schematic view showing the first nucleic acid extraction kit conveying module having nucleic acid extraction kits disposed thereon of the present disclosure, and FIG. 13 is a perspective side view of the sample transfer chamber of the present disclosure. The pre-processing procedures performed by the sample transfer chamber 10 mainly include opening and closing covers of the sampling tubes, identifying barcodes on the sampling tubes and transferring samples from the sampling tubes to the nucleic acid extraction kit. The sample transfer chamber 10 includes a sample transfer chamber gateway 101, a first air inlet 102, a first air outlet 103, a first bridging structure 40a, a sampling tube placement area 104, a first tip placement area 105, an area 106 for cover opening and closing and sample collection, a first nucleic acid extraction kit placement area 107, a cover opening and closing module 11, a sampling tube visual identification module 12, a lateral barcode identification module 13, a bottom barcode identification module (not shown), a first pipetting module 14, a first pipetting visual identification module 15, and a first nucleic acid extraction kit conveying module 16.

The sample transfer chamber gateway 101 is the path of the sample transfer chamber 10 to communicate with the external, for example, for moving in and out the sampling tubes 60, for moving in and out tips and related consumables, and for moving in the nucleic acid extraction kits 80. The sampling tube placement area 104 is used to place sampling tube racks with sampling tubes 60 accommodated therein, e.g., three sets of sampling tube racks with 96 sampling tubes can be placed. The first tip placement area 105 is used to place a tip rack 71 (such as a tip rack with 96 wells) and a mixed type tip rack 72 (such as a tip recycling container). The sampling tube placement area 104 further has a sensor disposed therein for sensing the placement of the sampling tube racks.

The bottom barcode identification module (not shown) is disposed under the sampling tube placement area 104 for identifying barcodes disposed at the bottoms of the sampling tubes 60. For example, a transparent window is disposed at the bottom of the sampling tube placement area 104, so the bottom barcode identification module can scan the barcodes. The lateral barcode identification module 13 is disposed at the area 106 for cover opening and closing and sample collection, and is used to scan the barcodes on the side walls of the sampling tubes as being moved to this area.

The cover opening and closing module 11 can be moved along X, Y and Z axes and between the sampling tube placement area 104 and the area 106 for cover opening and closing and sample collection. The cover opening and closing module 11 includes first clamping claws 111 for clamping the cover of the sampling tube 60 and being capable of rotating 360 degrees. In an embodiment, the cover opening and closing module 11 includes two sets of first clamping claws 111 for simultaneously clamping and grabbing two sampling tubes 60. The clamping claws can adapt to covers with different diameters. The sampling tube visual identification module 12 is combined and moved with the cover opening and closing module 11 and is used to identify the positions and the amounts of the sampling tubes 60 in the sampling tube placement area 104 for avoiding the placement error from interrupting the operation.

The area 106 for cover opening and closing and sample collection has a moving track (not shown) and second clamping claws (not shown) disposed therein. The moving track receives the sampling tube 60 and allows the sampling tube 60 to move between a cover opening and closing position and a sample collection position. The second clamping claws are used to clamp the side wall of the sampling tube 60 for stabilizing the sampling tube 60 as moving on the moving track, and also for cooperating with the first clamping claws 111, which clamp and rotate the cover, to complete the operation of cover opening and closing.

The first pipetting module 14 can be moved along X, Y and Z axes and among the first tip placement area 105, the area 106 for cover opening and closing and sample collection, and the first nucleic acid extraction kit placement area 107, so as to transfer the samples in the sampling tubes 60 to the nucleic acid extraction kits 80 placed in the first nucleic acid extraction kit placement area 107. The first pipetting module 14 includes a plurality of first pipettors 141 and a first pipetting liquid tray 142. In an embodiment, the number of the first pipettors 141 is implemented as two for individually and simultaneously transferring samples from two sampling tubes 60. The first pipetting liquid tray 142 is moved between an extended position and a retracted position. When in the extended position, the first pipetting liquid tray 142 is under the first pipettors 141 for receiving the dropped liquids during the first pipettors 141 transfer the samples to the nucleic acid extraction kit 80, so it is preferable that the first pipetting liquid tray 142 includes a retaining wall disposed at the periphery thereof to ensure that the liquids do not leak out. The first pipetting visual identification module 15 is combined and moved with the first pipetting module 14 and is used to identify the positions and amounts of the tips in the first tip placement area 105 and the positions and amounts of the nucleic acid extraction kits 80, for avoiding the placement error from interrupting the operation.

The first nucleic acid extraction kit placement area 107 is used to place the nucleic acid extraction kits 80. In an embodiment, the nucleic acid extraction kits 80 are placed on a carrying plate 81 first and then placed in the first nucleic acid extraction kit placement area 107. The first nucleic acid extraction kit placement area 107 can accommodate one to six nucleic acid extraction kits 80, wherein six nucleic acid extraction kits 80 can perform nucleic acid extraction operations of 96 samples. The first nucleic acid extraction kit conveying module 16 is used to convey the nucleic acid extraction kits 80 to the nucleic acid extraction chamber 20 and includes positioning components 161, belts 162 and sensors 163. The positioning components 161 are used to position the nucleic acid extraction kits 80, the belts 162 are used to drive the carrying plate 81 and the nucleic acid extraction kits 80 thereon to move, and the sensors 163 are used to sense whether the positioning of the nucleic acid extraction kits 80 is successful.

The first bridging structure 40a is used to connect with the second bridging structure 50b of the nucleic acid extraction chamber 20. The detailed structures and the connection manner are as described above and omitted.

The first air inlet 102 is disposed at the opposite side to the first bridging structure 40a, and the first air outlet 103 is disposed at the top of the chamber. Under this configuration, through the FFU disposed at the first air outlet 103, a first airflow AF1 in the sample transfer chamber 10 can be maintained in a single flowing direction from bottom to top, wherein the first airflow AF1 does not pass through the area above the nucleic acid extraction kits 80 after passing through the area above the uncovered sampling tubes 60, so as to effectively avoid the cross-contamination.

Before the operation of the sample transfer chamber 10, the operator opens the sample transfer chamber gateway 101 for placing the sampling tube racks with sampling tubes 60 in the sampling tube placement area 104, placing the tip rack 71 with tips and the mixed type tip rack 72 in the first tip placement area 105, and placing the nucleic acid extraction kit 80 in the first nucleic acid extraction kit placement area 107, and then closes the sample transfer chamber gateway 101, thereby completing the preparation before the automatic operation.

The automatic operation of the sample transfer chamber 10 is as follows. First, in the sampling tube placement area 104, the bottom barcode identification module scans the barcodes at the bottoms of the sampling tubes 60, and the cover opening and closing module 11 with the sampling tube visual identification module 12 is moved to the top of the sampling tube placement area 104. At this time, the sampling tube visual identification module 12 identifies the positions and amounts of the sampling tubes 60, and the first clamping claws 111 of the cover opening and closing module 11 grab the sampling tube 60 for moving the sampling tube 60 to the cover opening and closing position in the area 106 for cover opening and closing and sample collection. Then, in the cover opening and closing position, the lateral barcode identification module 13 scans the barcode on the side wall of the sampling tube 60, and further, the first clamping claws 111 clamp the cover of the sampling tube 60 and the second clamping claws clamp the side wall of the sampling tube 60 for cooperatively opening the cover, and then, the cover opening and closing module 11 with the cover is moved out of the area 106 for cover opening and closing and sample collection. Then, the uncovered sampling tube 60 is moved to the sample collection position along the moving track, and the first pipetting module 14 is moved to the first tip placement area 105 for the first pipettor 141 to combine with the tip and then moved to the uncovered sampling tube 60 for drawing up the sample. Then, the first pipetting module 14 is moved to the first nucleic acid extraction kit placement area 107 and the first pipetting liquid tray 142 is extended out during the movement for avoiding the liquid from dropping in the chamber. After the sample is released in the nucleic acid extraction kit 80, the first pipetting module 14 is moved to the mixed type tip rack 72 for ejecting the used tip and the first pipetting liquid tray 142 is extended out during the movement. Following, the sampling tube 60 is moved to the cover opening and closing position, and the cover opening and closing module 11 with the cover is moved to the top of the uncovered sampling tube 60, and then, the second clamping claws clamp the side wall of the sampling tube 60 and the first clamping claws 111 rotates the cover for cooperatively closing the cover. Finally, the first clamping claws 111 grab the sampling tube 60 and the cover opening and closing module 11 is moved to the sampling tube placement area 104 for placing the sampling tube 60 back to the sampling tube rack. At this point, one time of sample transfer is completed. The operations above are repeated until all samples in the sampling tubes are transferred to the nucleic acid extraction kit 80.

After all samples are transferred, the first partition board 43 of the first bridging structure 40a of the sample transfer chamber 10 is opened and the second partition board 53 of the second bridging structure 50b of the nucleic acid extraction chamber 20 is also opened, and at this time, the first nucleic acid extraction kit conveying module 16 is activated to convey the nucleic acid extraction kits 80 to the nucleic acid extraction chamber 20 via the connection channel 90. After conveying, the first partition board 43 and the second partition board 53 are closed. The nucleic acid extraction chamber 20 starts to perform the nucleic acid extraction operation, and the sample transfer chamber 10 starts to disinfect, such as, through the UV irradiation and the air replacement. After disinfection, the operator opens the sample transfer chamber gateway 101, moves out the first batch of sampling tubes and the used tips, and moves in the second batch of sampling tubes, the tips and the nucleic acid extraction kits, so as to complete the preparation for re-starting the pre-processing procedures for the second batch of samples.

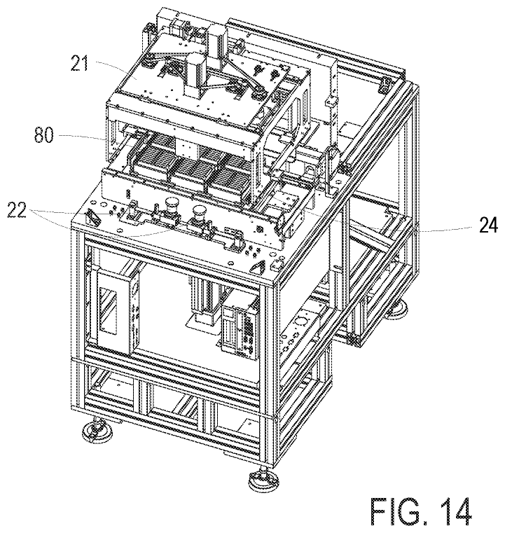

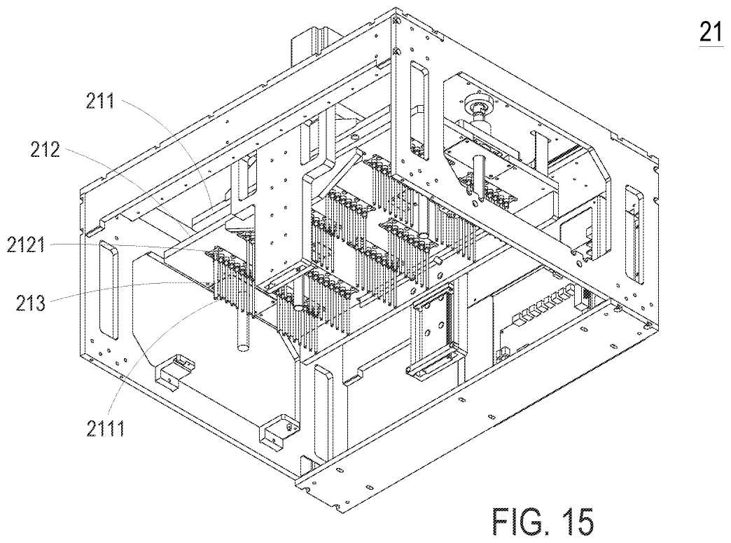

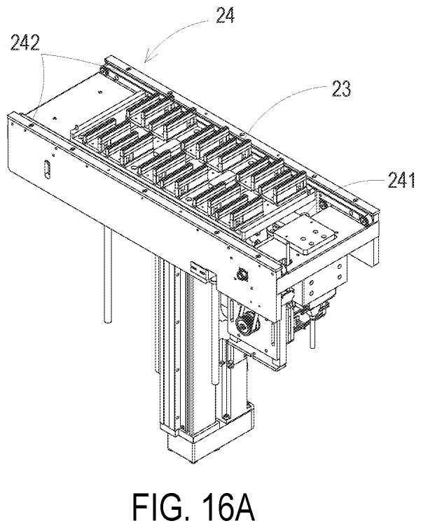

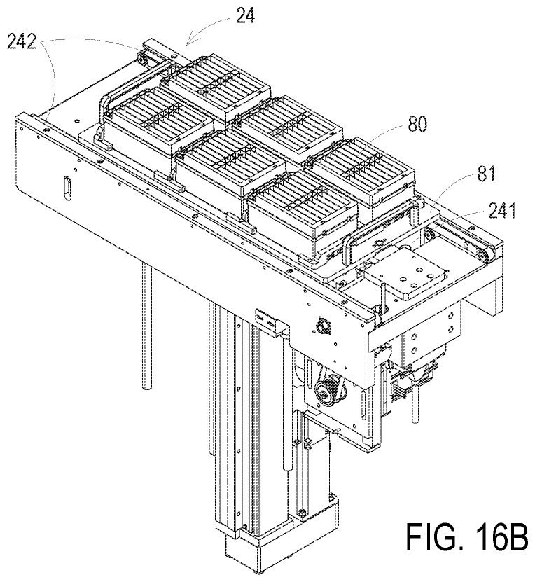

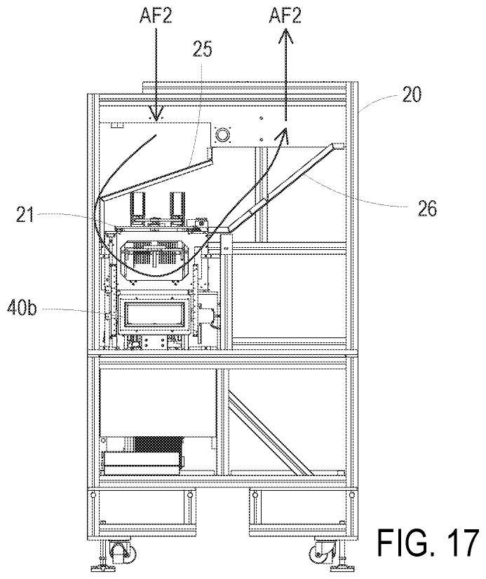

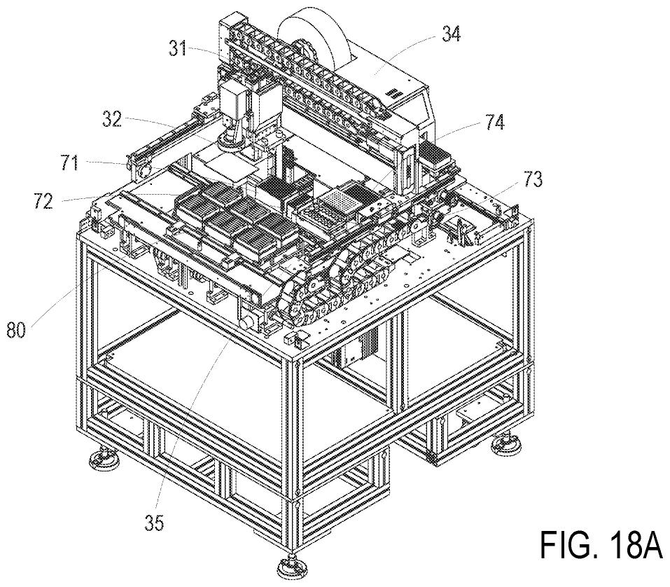

Please refer to FIG. 1, FIG. 2, FIGS. 4A-4B, FIG. 14, FIG. 15, FIGS. 16A-16B and FIG. 17. FIG. 14 is a schematic view showing portions of structures of the nucleic acid extraction chamber of the present disclosure, FIG. 15 is a schematic view showing a nucleic acid extraction module of the present disclosure, FIG. 16A is a schematic view showing a second nucleic acid extraction kit conveying module and a temperature controlling module of the present disclosure, FIG. 16B is schematic view showing the second nucleic acid extraction kit conveying module having nucleic acid extraction kits disposed thereon of the present disclosure, and FIG. 17 is a perspective side view of the nucleic acid extraction chamber of the present disclosure. The nucleic acid extraction chamber 20 is used to perform the nucleic acid extraction operation, such as the magnetic beads-based nucleic acid extraction operation. The nucleic acid extraction chamber 20 includes a second air inlet 201, a second air outlet 202, a second nucleic acid extraction kit placement area 203, the first bridging structure 40b, the second bridging structure 50b, a nucleic acid extraction module 21, a magnetic rod sleeve visual identification module 22, a temperature controlling module 23, a second nucleic acid extraction kit conveying module 24, a first deflector 25 and a second deflector 26.

The second nucleic acid extraction kit placement area 203 is used to place the nucleic acid extraction kits 80, such as six nucleic acid extraction kits 80. The nucleic acid extraction module 21 is used to perform the nucleic acid extraction operations of the samples and includes a magnetic rod holder 211 for mounting a plurality of magnetic rods 2111 thereon, and a magnetic rod sleeve connector holder 212 for mounting a plurality of magnetic rod sleeve connectors 2121 thereon. The magnetic rod sleeve connectors 2121 are used to connect to the magnetic rod sleeves 213 pre-set in the nucleic acid extraction kits 80, wherein corresponding to each nucleic acid extraction kit 80, two rows of 8 magnetic rods and two rods of 8 magnetic rod sleeve connectors are provided. The magnetic rod sleeve visual identification module 22 is used to identify the connections of the magnetic rod sleeve connectors 2121 on the nucleic acid extraction module 21 with the magnetic rod sleeves 213 in the nucleic acid extraction kits 80 for avoiding the connection error from interrupting the operation or from causing contamination. The temperature controlling module 23 is implemented to match every well of the nucleic acid extraction kit 80, e.g., the temperature controlling module 23 can be nestled up against the bottom of each well so as to provide the required temperature variation during the nucleic acid extraction operation.

The second nucleic acid extraction kit conveying module 24 is used to convey the nucleic acid extraction kits 80 to the assay setup chamber 30 and includes positioning components 241, belts 242 and sensors (not shown). The positioning components 241 are used to position the nucleic acid extraction kits 80s, the belts 242 are used to drive the nucleic acid extraction kits 80 to move, and the sensors are used to sense whether the positioning of the nucleic acid extraction kits 80 is successful. Moreover, the second nucleic acid extraction kit conveying module 24 includes a Z axis lifting mechanism which can lift the temperature controlling module 23 and the nucleic acid extraction kits 80 to a height for the nucleic acid extraction module 21 to perform the nucleic acid extraction operation.

The second bridging structure 50b is used to connect with the first bridging structure 40a of the sample transfer chamber 10, and the first bridging structure 40b is used to connect with the second bridging structure 50c of the assay setup chamber 30. The detailed structures and the connection manner are as described above and omitted.

The second air inlet 201 is disposed at the front part of the top of the nucleic acid extraction chamber 20, namely, at a position approximately corresponding to the nucleic acid extraction module 21, and the second air outlet 202 is disposed at the rear part of the top of the chamber. The first deflector 25 is disposed under the second air inlet 201 and separates the second air inlet 201 from the second air outlet 202. The first deflector 25 is arranged to have a gradually downward tendency from the rear side of the chamber to the front side thereof for guiding a second airflow AF2 to flow downwardly into the second nucleic acid extraction kit placement area 203 from the front side of the chamber. The second deflector 26 is disposed under the second air outlet 202, at the rear side of the first deflector 25 and at a Z axis position higher than the nucleic acid extraction module 21, and is arranged to have a gradually upward tendency from the front side of the chamber to the rear side thereof, so the second airflow AF2 which is driven by the FFU at the second air outlet 202 can be guided by the second deflector 26 to move upwardly from the second nucleic acid extraction kit placement area 203 and discharged through the second air outlet 202. Under this configuration, the second airflow AF2 in the nucleic acid extraction chamber 20 can be maintained in a single flowing direction, wherein the second airflow AF2 passes through the nucleic acid extraction kits 80 in a downward direction for avoiding aerosols generated during the nucleic acid extraction operations from moving with the airflow to cause the cross contamination between different wells of the nucleic acid extraction kits 80. Furthermore, the second airflow AF2 downwardly passes through the area for performing the nucleic acid extraction operation and then moves upwardly to pass the second air outlet 202, so it can ensure that the air in the chamber is replaced effectively.

The operation of nucleic acid extraction chamber 20 is as follows. The second nucleic acid extraction kit conveying module 24 receives the nucleic acid extraction kits 80 having samples transferred therein from the sample transfer chamber 10 and positions thereof in the second nucleic acid extraction kit placement area 203. Then, the Z axis lifting mechanism lifts up the temperature controlling module 23 to contact the bottoms of the wells of the nucleic acid extraction kits 80, and further lifts up the nucleic acid extraction kits 80 to the height for performing the nucleic acid extraction operation. The procedures for performing the nucleic acid extraction are as follows.

The magnetic rod sleeve connector holder 212 is moved downwardly to connect the magnetic rod sleeve connectors 2121 thereon with the magnetic rod sleeves 213 pre-set in the nucleic acid extraction kits 80. Then, through the up and down movements of the magnetic rod sleeve connector holder 212, the magnetic rod sleeves 213 connected with the magnetic rod sleeve connectors 2121 can sequentially perform the steps of magnetic beads-based nucleic acid extraction in each well of the nucleic acid extraction kits 80, e.g., the steps of cell lysis, nucleic acid adsorption, washing and elution. During these processes, when there is a need to absorb the magnetic beads, the magnetic rod holder 211 is moved downwardly to make the magnetic rods 2111 mounted thereon to pass through the magnetic rod sleeve connectors 2121 and arrive the bottom portions of the magnetic rod sleeves 213, so that the magnetic beads in the wells of the nucleic acid extraction kits 80 are absorbed by the magnetic rods 2111, and the magnetic rod holder 211 and the magnetic rod sleeve connector holder 212 are moved together to transfer the absorbed magnetic beads to the next wells. Then, the magnetic rod holder 211 is moved upwardly to separate the magnetic rods 2111 from the magnetic beads and thus release the magnetic beads, and at the same time, the magnetic rod sleeves 213 are driven by the magnetic rod sleeve connector holder 212 to have up-and-down vibrations for speeding up the dispersion of magnetic beads to be mixed with the liquid. After the nucleic acid extraction operation is completed, the Z axis lifting mechanism lowers down the temperature controlling module 23 and the nucleic acid extraction kits 80, and the nucleic acid extraction kits 80 are back to the height corresponding to the first bridging structure 40b. Then, the first partition board 43 of the first bridging structure 40b of the nucleic acid extraction chamber 20 is opened and the second partition board 53 of the second bridging structure 50c of the assay setup chamber 30 is also opened, and at this time, the second nucleic acid extraction kit conveying module 24 is activated to convey the nucleic acid extraction kits 80 to the assay setup chamber 30. After conveying, the first partition board 43 and the second partition board 53 are closed. The assay setup chamber 30 starts to perform the assay setup procedures and the nucleic acid extraction chamber 20 starts to disinfect. After disinfection, the nucleic acid extraction chamber 20 once again receives the nucleic acid extraction kits 80 from the sample transfer chamber 10 and re-starts the nucleic acid extraction operation.

Here, through cooperating the nucleic acid extraction kits 80 having the pre-set magnetic rod sleeves 213 and the pre-loaded reagents with the capability of the nucleic acid extraction module 21 to automatically connect and disconnect with the magnetic rod sleeves 213, the nucleic acid extraction operation in the nucleic acid extraction chamber 20 can be completed automatically, namely, there is no need of manual intervention during the entire process including moving the nucleic acid extraction kits 80 into the nucleic acid extraction chamber 20, performing the nucleic acid extraction operation and moving the nucleic acid extraction kits 80 out of the nucleic acid extraction chamber 20. Moreover, in the nucleic acid extraction chamber 20, the nucleic acid extraction operations of all samples in the nucleic acid extraction kits 80 are performed simultaneously and individually, for example, when six nucleic acid extraction kits 80 are moved in, the nucleic acid extraction operations of 96 samples are performed at the same time.

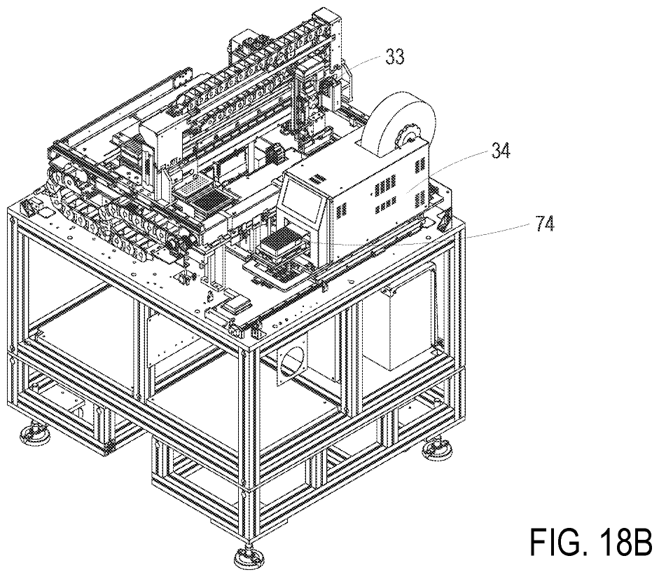

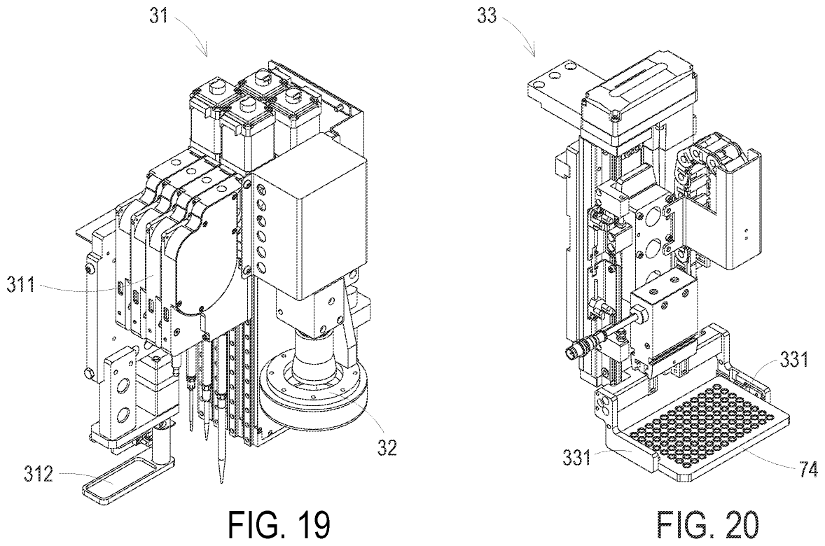

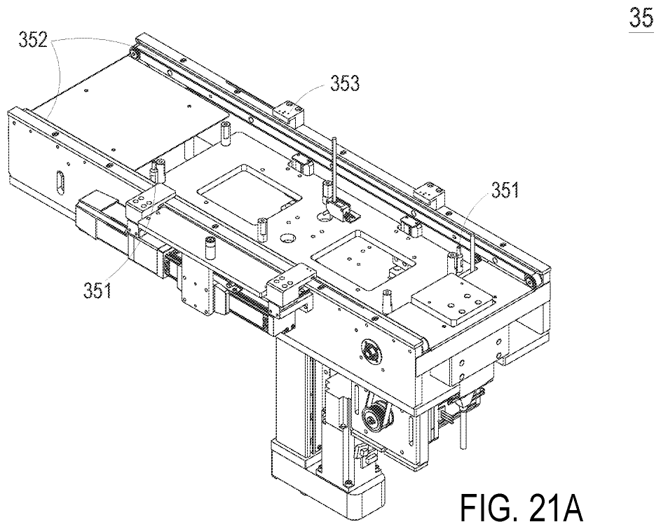

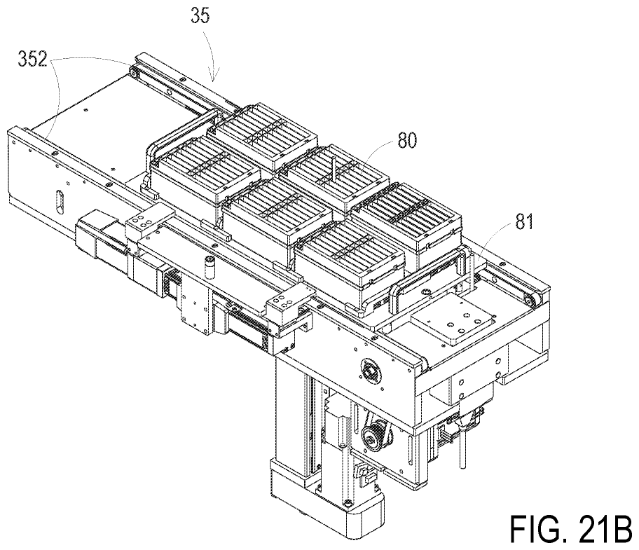

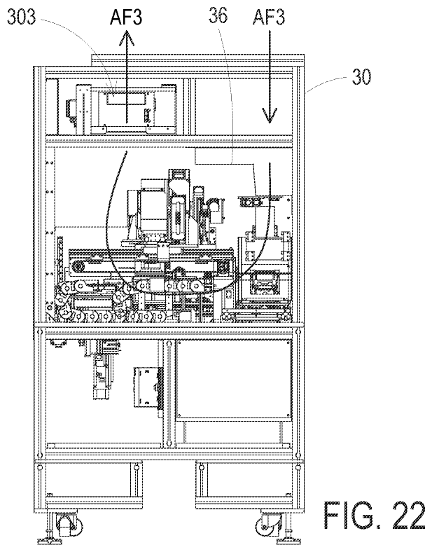

Please refer to FIG. 1, FIG. 2, FIGS. 5A-5B, FIGS. 18A-18B, FIG. 19, FIG. 20, FIGS. 21A-21B and FIG. 22. FIG. 18A is a schematic view showing portions of structures of the assay setup chamber of the present disclosure, FIG. 18B is a schematic view showing portions of structures of the assay setup chamber of the present disclosure from another view angle, FIG. 19 is a schematic view showing a second pipetting module and a second pipetting visual identification module of the present disclosure, FIG. 20 is a schematic view showing an assay plate conveying module of the present disclosure, FIG. 21A is a schematic view showing a third nucleic acid extraction kit conveying module of the present disclosure, FIG. 21B is a schematic view showing the third nucleic acid extraction kit conveying module having nucleic acid extraction kits disposed thereon of the present disclosure, and FIG. 22 is a perspective side view of the assay setup chamber of the present disclosure. The pre-processing procedures performed by the assay setup chamber 30 mainly include preparing reagents and transferring nucleic acid extracts. The assay setup chamber 30 includes an assay setup chamber gateway 301, a third air inlet 302, a third air outlet 303, the second bridging structure 50c, a reagent placement area 304, an assay plate placement area 305, a second tip placement area 306, a third nucleic acid extraction kit placement area 307, a second pipetting module 31, a second pipetting visual identification module 32, a reagent cooling module (not shown), an assay plate cooling module (not shown), an assay plate conveying module 33, an assay plate sealing module 34, a third nucleic acid extraction kit conveying module 35 and a third deflector 36.

The assay setup chamber gateway 301 is the path of the assay setup chamber 30 to communicate with the external, for example, for moving in and out reagents, for moving in and out tips and related consumables, and for moving out the nucleic acid extraction kits 80. The reagent placement area 304 is used to place a reagent plate 73 for accommodating reagent tubes and reagent mixing tubes and also has the reagent cooling module (not shown) disposed therein to keep the reagents at a low temperature. In an embodiment, the reagent placement area 304 has 12 reagent tubes and 4 reagent mixing tubes disposed therein. The assay plate placement area 305 is used to place assay plates 74 and also has the assay plate cooling module (not shown) disposed therein. In an embodiment, the assay plate placement area 305 has 2 assay plates 74 disposed therein. The second tip placement area 306 is used to place the tip rack 71 and the mixed type tip rack 72.

The second pipetting module 31 can be moved along X, Y and Z axes and among the reagent placement area 304, the assay plate placement area 305, the second tip placement area 306 and the third nucleic acid extraction kit placement area 307, so as to deploy the reagents, dispense the reagent to the assay plates 74, and transfer the nucleic acid extracts from the nucleic acid extraction kits 80 to the assay plates 74. The second pipetting module 31 includes a plurality of second pipettors 311 and a second pipetting liquid tray 312. In an embodiment, the number of the second pipettors 311 is implemented to be four for individually and simultaneously transferring four nucleic acid extracts. The second pipetting liquid tray 312 is moved between an extended position and a retracted position. When in the extended position, the second pipetting liquid tray 312 is under the second pipettors 311 for receiving the dropped liquids during the second pipettors 311 transfer the reagents/the nucleic acid extracts, so it is preferable that the second pipetting liquid tray 312 includes a retaining wall disposed at the periphery thereof to ensure the liquids do not leak out. The second pipetting visual identification module 32 is combined and moved with the second pipetting module 31 and is used to identify the positions and amounts of the tips and the reagent tubes for avoiding the placement error from interrupting the operation.

The assay plate conveying module 33 can be moved along X, Y and Z axes. The assay plate conveying module 33 further includes third clamping claws 331 for clamping the assay plate 74, so the assay plate 74 with the dispensed reagents and the transferred nucleic acid extracts can be moved from the assay plate placement area 305 to the assay plate sealing module 34 to perform the film sealing procedure. After completing the film sealing procedure, the assay plate 74 is moved back to the assay plate placement area 305 by the assay plate conveying module 33. In an embodiment, the third clamping claws 331 have a sensor (not shown) disposed thereon for ensuring whether the assay plate 74 is clamped and placed correctly. The assay plate sealing module 34 is used to perform the film sealing procedure to the assay plate 74 with the dispensed reagents and the transferred nucleic acid extracts so as to avoid the contamination before the nucleic acid detection.

The third nucleic acid extraction kit placement area 307 is used to place the nucleic acid extraction kits 80. The third nucleic acid extraction kit conveying module 35 is used to receive the nucleic acid extraction kits 80 from the nucleic acid extraction chamber 20 and includes positioning components 351, belts 352 and sensors 353. The positioning components 351 are used to position the nucleic acid extraction kits 80, the belts 352 are used to drive the nucleic acid extraction kits 80 to move, and the sensors 353 are used to sense whether the positioning of the nucleic acid extraction kits 80 is successful.

The second bridging structure 50c is used to connect with the first bridging structure 40b of the nucleic acid extraction chamber 20. The detailed structures and the connection manner are as described above and omitted.

The third air inlet 302 is disposed at the rear part of the top of the assay setup chamber, namely, at a position approximately above the assay plate sealing module 34, and the third air outlet 303 is disposed at the front part of the top of the chamber, namely, at a position approximately above the third nucleic acid extraction kit placement area 307. The third deflector 36 is disposed between the third air inlet 302 and the third air outlet 303 for separating the flow-in airflow from the flow-out airflow. Under this configuration, a third airflow AF3 in the assay setup chamber 30 can be maintained in a single flowing direction, wherein the third airflow AF3 downwardly passes through the reagent placement area 304, the assay plate placement area 305 and the second tip placement area 306 first, then passes through the third nucleic acid extraction kit placement area 307, and is discharged through the third air outlet 303. Therefore, the airflow passing through the nucleic acid extraction kits 80 will not pass through the area where the reagent plate 73 and the assay plates 74 are placed, thereby effectively avoiding the cross-contamination.

Before the operation of the assay setup chamber 30, the operator opens the assay setup chamber gateway 301 for placing the reagent plate 73 with the reagent tubes and the reagent mixing tubes in the reagent placement area 304, placing the tip rack 71 with tips and the mixed-type tip rack 72 in the second tip placement area 306, and placing the assay plates 74 in the assay plate placement area 305, and then closes the assay setup chamber gateway 301, thereby completing the preparation before the automatic operation.

The automatic operation of the assay setup chamber 30 is as follows. First, the second pipetting module 31 together with the second pipetting visual identification module 32 are moved among the reagent placement area 304, the assay plate placement area 305 and the second tip placement area 306 for utilizing the second pipettors 311 to deploy the reagents and to dispense the deployed reagent to the assay plates 74 until each well of the assay plates 74 is loaded with the deployed reagent. After the reagent loading, the assay setup chamber 30 is in a standby state for waiting the nucleic acid extraction operation in the nucleic acid extraction chamber 20 to be completed. Then, the third nucleic acid extraction kit conveying module 35 receives the nucleic acid extraction kits 80 with the nucleic acid extracts from the nucleic acid extraction chamber 20 and positions thereof in the third nucleic acid extraction kit placement area 307. Following, the second pipetting module 31 together with the second pipetting visual identification module 32 are moved among the assay plate placement area 305, the second tip placement area 306 and the third nucleic acid extraction kit placement area 307 for utilizing the second pipettors 311 to transfer the nucleic acid extracts to the assay plates 74, which already have the deployed reagent loaded therein, until the nucleic acid extracts in all wells of the nucleic acid extraction kits 80 are transferred to the assay plates 74. During the second pipetting module 31 is performing the deployment of reagents and the transfers of nucleic acid extracts, the second pipetting liquid tray 312 is extended out for avoiding the liquid from dropping in the chamber. Finally, the assay plate conveying module 33 is moved to the assay plate placement area 305 to clamp and move the assay plate 74, which is loaded with the deployed reagent and the nucleic acid extracts, to the assay plate sealing module 34, and after the film sealing procedure is completed, the assay plate conveying module 33 moves the assay plate 74 back to the assay plate placement area 305.

Then, the operator opens the assay setup chamber gateway 301 for moving out the film sealed assay plates 74 and the used tips, and supplying the tips, the assay plates and the reagents, and closes the assay setup chamber gateway 301 for performing the disinfection. After the disinfection, the assay setup chamber 30 re-starts to operate and once again receives the nucleic acid extraction kits 80 from the nucleic acid extraction chamber 20.

In addition, the sample transfer chamber 10 and the assay setup chamber 30 respectively further have an image identification module (not shown) disposed therein for identifying the positions and amounts of all materials in the chamber after the loading and unloading of the materials by the operator, thereby avoiding the shortage of materials from interrupting the operation of the chamber.

In conclusion, the combinable nucleic acid pre-processing apparatus of the present disclosure includes advantages as follows:

High chamber independency: Through disposing the bridging modules, each chamber can form an independent space respectively and also can be used to independently process different batches of samples without influencing each other, and when an abnormality occurs, only the abnormal chamber has to be paused and other chambers still can operate normally.

High throughput: After completing the pre-processing procedures of a current batch of samples and transmitting thereof to the next chamber, the chamber can immediately perform the pre-processing procedures for a next batch of samples, so the processing speed of the entire apparatus can be significantly improved, thereby increasing the throughput per unit time.

Low cross-contamination possibility: Through arranging the positions of the air inlet, the air outlet and the deflector(s), an airflow direction suitable for each chamber can be achieved for effectively reducing the cross-contamination between different samples in the same batch; Moreover, each chamber is disinfected after performing the procedures of each batch of samples, so the cross-contamination between different batch of samples can be effectively reduced.

Easy transportation and installation: The apparatus can be disassembled into independent chambers for transportation and reassembled at the desired location, and the assembling of the chambers can be easily achieved by combining the engaging members of the bridging modules, which is extremely convenient.

Fully automated process: All procedures after obtaining the samples and before performing the PCR detection, including identifying barcodes on the sampling tubes, opening and closing covers of the sampling tubes, transferring samples from the sampling tubes, performing the nucleic acid extraction operation, preparing reagents, and transferring nucleic acid extracts, are performed automatically, which significantly reduces the manual requirements.

High intelligence: The visual identification is utilized to ensure that, in each chamber, the positions and amounts of the materials and the movements of each module are correct for avoiding the incorrect quantities and/or positions and movements from interrupting the operation and wasting time.

While the disclosure has been described in terms of what is presently considered to be the most practical and preferred embodiments, it is to be understood that the disclosure needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A combinable nucleic acid pre-processing apparatus, comprising:
a sample transfer chamber comprising a cover opening and closing module and a first pipetting module for transferring a sample from a sampling tube to a nucleic acid extraction kit;
a nucleic acid extraction chamber comprising a nucleic acid extraction module for performing a nucleic acid extraction operation on the sample in the nucleic acid extraction kit so as to obtain a nucleic acid extract;
an assay setup chamber comprising a second pipetting module for deploying a reagent and adding the deployed reagent and the nucleic acid extract into an assay plate; and
at least two bridging modules respectively disposed between the sample transfer chamber and the nucleic acid extraction chamber and between the nucleic acid extraction chamber and the assay setup chamber,
wherein the sample transfer chamber, the nucleic acid extraction chamber and the assay setup chamber are separated from each other and operated independently, and the sample transfer chamber, the nucleic acid extraction chamber and the assay setup chamber are further connected through the at least two bridging modules for achieving a sequential movement of the nucleic acid extraction kit in the sample transfer chamber, the nucleic acid extraction chamber and the assay setup chamber in a duration of nucleic acid pre-processing.

2. The combinable nucleic acid pre-processing apparatus according to claim 1, wherein the sample transfer chamber, the nucleic acid extraction chamber and the assay setup chamber individually perform nucleic acid pre-processing procedures of different batches of samples simultaneously.

3. The combinable nucleic acid pre-processing apparatus according to claim 1, wherein each bridging module comprises a first bridging structure and a second bridging structure, respectively disposed on adjacent sides of adjacent two chambers, and by combining the first bridging structure and the second bridging structure, the adjacent two chambers are connected.

4. The combinable nucleic acid pre-processing apparatus according to claim 3, wherein the first bridging structure comprises a first engaging member, a first opening, a first partition board and a first sliding rail, and the first partition board moves along the first sliding rail to open or close the first opening, and wherein the second bridging structure comprises a second engaging member, a second opening, a second partition board and a second sliding rail, and the second partition board moves along the second sliding rail to open or close the second opening.

5. The combinable nucleic acid pre-processing apparatus according to claim 4, wherein the first engaging member and the second engaging member are engaged to form a connection channel between the adjacent two chambers for the nucleic acid extraction kit to pass therethrough, the first partition board and the second partition board are located at two opposite ends of the connection channel, and through closing and opening the first opening and the second opening, the adjacent two chambers are mutually separated or connected.

6. The combinable nucleic acid pre-processing apparatus according to claim 4, wherein the first bridging structure comprises a first protrusion board for forming an inverted trapezoidal shape space around the first opening, the first partition board comprises a second protrusion board in an inverted trapezoidal shape, airtight elements are disposed at a periphery of the second protrusion board, and when the first partition board is moved downwardly, the second protrusion board enters the inverted trapezoidal shape space, and wherein the second bridging structure comprises a third protrusion board for forming an inverted trapezoidal shape space around the second opening, the second partition board comprises a fourth protrusion board in an inverted trapezoidal shape, airtight elements are disposed at a periphery of the fourth protrusion board, and when the second partition board is moved downwardly, the fourth protrusion board enters the inverted trapezoidal shape space.

7. The combinable nucleic acid pre-processing apparatus according to claim 1, wherein the sample transfer chamber comprises a first nucleic acid extraction kit conveying module for conveying the nucleic acid extraction kit to the nucleic acid extraction chamber.

8. The combinable nucleic acid pre-processing apparatus according to claim 1, wherein the sample transfer chamber comprises a first air inlet, a first air outlet and a fan filter unit disposed at the first air outlet for maintaining a first airflow entering the sample transfer chamber in a single direction, and the first airflow does not pass through an area where the nucleic acid extraction kit is placed after passing through an uncovered sampling tube, and wherein the first air inlet and the first air outlet respectively have high efficiency particulate air filters disposed thereon.

9. The combinable nucleic acid pre-processing apparatus according to claim 1, wherein the nucleic acid extraction module comprises a magnetic rod holder, a plurality of magnetic rods mounted on the magnetic rod holder, a magnetic rod sleeve connector holder, and a plurality of magnetic rod sleeve connectors mounted on the magnetic rod sleeve connector holder for performing a magnetic beads-based nucleic acid extraction operation.

10. The combinable nucleic acid pre-processing apparatus according to claim 1, wherein the nucleic acid extraction chamber comprises a second nucleic acid extraction kit conveying module for receiving the nucleic acid extraction kit from the sample transfer chamber and for conveying the nucleic acid extraction kit to the assay setup chamber.

11. The combinable nucleic acid pre-processing apparatus according to claim 1, wherein the nucleic acid extraction chamber comprises a temperature controlling module for matching with each well of the nucleic acid extraction kit so as to provide a temperature variation required during the nucleic acid extraction operation.

12. The combinable nucleic acid pre-processing apparatus according to claim 1, wherein the nucleic acid extraction chamber comprises a second air inlet, a second air outlet, a first deflector, a second deflector and a fan filter unit disposed at the second air outlet for maintaining a second airflow entering the nucleic acid extraction chamber in a single direction, and the second airflow downwardly passes through an area for performing the nucleic acid extraction operation, and wherein the second air inlet and the second air outlet respectively have high efficiency particulate air filters disposed thereon.

13. The combinable nucleic acid pre-processing apparatus according to claim 1, wherein the assay setup chamber comprises a third nucleic acid extraction kit conveying module for receiving the nucleic acid extraction kit from the nucleic acid extraction chamber.

14. The combinable nucleic acid pre-processing apparatus according to claim 1, wherein the assay setup chamber comprises an assay plate conveying module and an assay plate sealing module, the assay plate conveying module conveys the assay plate with the added reagent and the added nucleic acid extract to the assay plate sealing module, and the assay plate sealing module performs a film sealing procedure to the assay plate.

15. The combinable nucleic acid pre-processing apparatus according to claim 1, wherein the assay setup chamber comprises a third air inlet, a third air outlet, a third deflector and a fan filter unit disposed at the third air outlet for maintaining a third airflow entering the assay setup chamber in a single direction, and the third airflow flows toward an area where the nucleic acid extraction kit is placed, and wherein the third air inlet and the third air outlet respectively have high efficiency particulate air filters disposed thereon.
